(19) United States
(12) Patent Application Publication
(10) Pub. No.: US 2026/0182878 A1
(43) Pub. Date: Jul. 2, 2026

(54) SYSTEM AND METHOD FOR ASSESSMENT OF COGNITIVE PERFORMANCE

(71) Applicant: Southwest Research Institute, San Antonio, TX (US)

(72) Inventors: Katelyn E. FRY-HILDERBRAND, San Antonio, TX (US); Koen F. FLORES, San Antonio, TX (US); Brian C. SWENSON, San Antonio, TX (US); Kase James SAYLOR, San Antonio, TX (US)

(21) Appl. No.: 19/358,654

(22) Filed: Oct. 15, 2025

Related U.S. Application Data
(60) Provisional application No. 63/711,324, filed on Oct. 24, 2024.

(51) Int. Cl. A61B 5/16; A61B 5/00

(52) U.S. Cl. CPC A61B 5/165; A61B 5/163; A61B 5/4803; A61B 5/7203; A61B 5/7267; A61B 2562/06

(57) ABSTRACT

In an approach to assessment of cognitive performance, a method includes attaching a plurality of portable physiological sensors to a body of a user; collecting data from the plurality of portable physiological sensors; processing the data using a cognitive performance algorithm and a pre-trained subject-specific model; and reporting a results of the cognitive performance algorithm.

### CROSS-REFERENCE TO RELATED APPLICATIONS

The present application claims the benefit of the filing date of U.S. Provisional Application Ser. No. 63/711,324, filed Oct. 24, 2024, the entire teachings of which application is hereby incorporated herein by reference.

### FIELD

The present disclosure relates generally to predicting cognitive performance and, more particularly, to a system and method for assessment of cognitive performance.

### BACKGROUND

Cognitive load describes the amount of working memory resources that are used at a given time and is a measure of how hard the brain is working on a current task. Working memory not only stores information short-term, but it enables the generation of new thoughts and ideas. Cognitive overload occurs when a working memory is overloaded, which negatively affects learning, planning, problem solving, and decision making. As a result, tools to assess cognitive load are desirable for detecting instances of cognitive overload.

Psychophysiological measurements such as eye tracking and electrocardiogram have been used to objectively assess cognitive load. However, current implementations of these methods are largely constrained with respect to the task space in which they can be applied. These methods are largely designed for use in a controlled environment where the subject is stationary and there is ample time for setup. Existing assessments cannot be used to estimate cognitive performance or to assess brain health in an operational environment. Additionally, these methods tend to rely on generalized performance prediction models, trained on population data rather than individualized data, decreasing the accuracy of these models for individualized performance prediction

### BRIEF DESCRIPTION OF THE DRAWINGS

Reference should be made to the following detailed description which should be read in conjunction with the following figures, wherein like numerals represent like parts.

FIG. 1 is a functional block diagram illustrating a system for assessment of cognitive performance consistent with the present disclosure.

FIG. 2 shows a schematic diagram of the system of FIG. 1 to predict an individual's cognitive performance consistent with the present disclosure.

FIG. 3 is a flowchart diagram depicting operations for one illustrative example embodiment of the process for assessment of cognitive performance on the system of FIG. 1, consistent with the present disclosure.

FIG. 4 is a flowchart diagram depicting operations for one illustrative example embodiment of the cognitive performance algorithm for the process of FIG. 3, consistent with the present disclosure.

FIG. 5 is a flowchart diagram depicting operations for one illustrative example embodiment of the feature selection process for the process of FIG. 3, consistent with the present disclosure.

FIG. 6 depicts a block diagram of components of the computing device executing the process for assessment of cognitive performance within the system of FIG. 1, consistent with the present disclosure.

### DETAILED DESCRIPTION

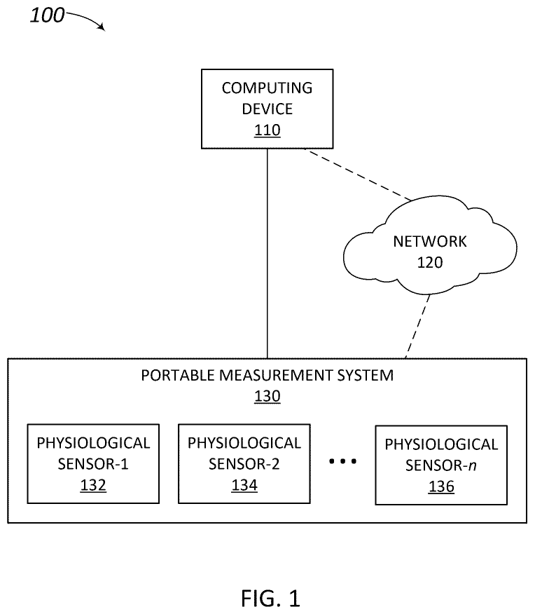

FIG. 1 is a functional block diagram illustrating a system 100, suitable for assessment of cognitive performance consistent with the present disclosure. The term “distributed” as used herein describes a computer system that includes multiple, physically distinct devices that operate together as a single computer system. FIG. 1 provides only an illustration of one implementation and does not imply any limitations with regard to the environments in which different embodiments may be implemented. Many modifications to the depicted environment may be made by those skilled in the art without departing from the scope of the disclosure as recited by the claims.

System 100 includes computing device 110 optionally connected to network 120. Network 120 can be, for example, a telecommunications network, a personal area network (PAN), such as Bluetooth, a local area network (LAN), a wide area network (WAN), such as the Internet, or a combination of the three, and can include wired, wireless, or fiber optic connections. In general, network 120 can be any combination of connections and protocols that will support communications between computing device 110 and other computing devices (not shown) within system 100.

In an embodiment, computing device 110 can be a standalone computing device, a management server, a web server, a mobile computing device, or any other electronic device or computing system capable of receiving, sending, and processing data. In another embodiment, computing device 110 can represent a server computing system utilizing multiple computers as a server system, such as in a cloud computing environment. In yet another embodiment, computing device 110 represents a computing system utilizing clustered computers and components (e.g., database server computers, application server computers) that act as a single pool of seamless resources when accessed within system 100.

In an embodiment, system 100 may include a portable measurement system 130. The portable measurement system 100 includes one or more physiological sensors, which may include, but are not limited to, an eye tracking camera to measure eye microsaccades, a heart rate sensor to measure heart rate variability and a microphone to measure voice audio waveforms. In an embodiment, the portable measurement system 130 includes n physiological sensors, where n is any whole number greater than 0, such as physiological sensor-1 132, physiological sensor-2 134, through physiological sensor-n 136. In some embodiments, the one or more physiological sensors may be wearable sensors or other portable sensors. In an embodiment, the physiological sensors, physiological sensor-1 132, physiological sensor-2 134, through physiological sensor-n 136, may include at least one of a chest-strap heartrate sensor, a near infrared vision camera, a head worn headset with microphone, or combinations thereof.

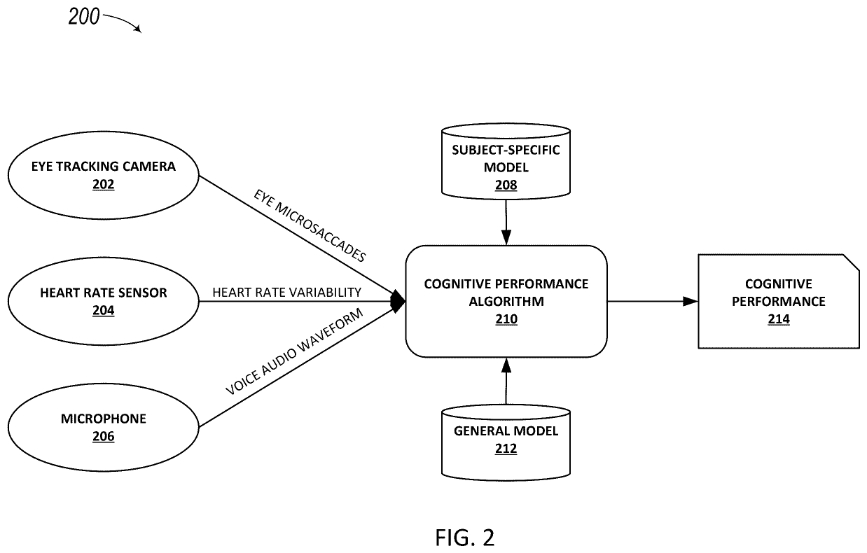

FIG. 2 shows a schematic diagram of a system 200 to predict an individual's cognitive performance. A plurality of physiological inputs are measured at 202, 204, and 206 when cognitive loading is applied, as described above in FIG. 1.

As may be appreciated, an individual is administered cognitive loading tasks, which may include reading working memory, rotational working memory, remote associates testing, and Raven's Progressive Matrices testing. The measurements are then introduced into a processor, such as computing device 110 from FIG. 1, utilizing a cognitive performance algorithm 210, which has been trained via use of an individual or subject-specific model 208 and general model 212 which then outputs a prediction of the individuals cognitive performance 214. A cognitive performance algorithm herein refers to a systematic process or computational model designed to identify human cognitive abilities, using methods from machine learning and artificial intelligence. The subject-specific model 208 is trained on data from a specific subject, while the general model 212 is trained on data from a general population. The general model 212 can provide predictions of an individual's performance, though refining this model with subject-specific data is used to improve overall prediction efficiency.

Accordingly, as shown in FIG. 2, the system predicts cognitive performance from a plurality of psychophysiological measurements. The system, when integrated into a portable measurement system, such as the portable measurement system 130 from FIG. 1, provides relatively accurate assessments of cognitive load and cognitive performance in operational settings as compared to systems requiring controlled, stationary environments. The system herein offers significant potential for applications in diverse fields, such as field research, military operations, and relatively high-stress work environments, where real-time cognitive assessment and insight are critical.

One application of the disclosed system may be in the field of decision-making in high pressure scenarios which may include, but are not limited to, military personnel, air traffic controllers, first responders, bomb disposal personnel, etc. In these scenarios, the physical state of the individual may affect their decision-making ability. Therefore, the system creates a baseline cognitive capability for the individual and can determine the relationship between fatigue and cognitive performance for the individual.

Another application of the disclosed system may be to use the cognitive baseline determined for an individual to check for a neurological injury.

An additional application of the disclosed system may be to test the cognitive capability for key personnel for making decisions in cognitively taxing situations. Some examples of these key personnel may include, but are not limited to, air traffic controllers, first responders in disaster situations, and bomb disposal personnel.

As can be observed in FIG. 2, the system therefore analyzes physiological data (e.g., eye tracking, speech, and heart rate variability) collected while subjects undertake cognitive loading tasks to predict cognitive performance, thereby providing insights into cognitive load and potential instances of cognitive overload. The system utilizes two complementary models: the general model 212 and the subject-specific model 208.

The general model 212 uses measurements gathered from lab-based sensors. The sensors therefore preferably include electrocardiogram (ECG), electrodermal activity (EDA), and the eye tracking was preferably done using an eye tracking system. These sensors, often more precise but less practical for field use, allow for a deeper understanding of physiological patterns. The general model 212 captures the broader relationships between these physiological signals and cognitive performance across different individuals.

In one illustrative embodiment, the sampling frequency for ECG, and EDA may be 1000 Hz and the sampling frequency for eye tracking may be 500 Hz. Speech sampling could also be conducted at a sampling of 44.1 kHz. In some other embodiments, other sampling frequencies may be used. It should therefore be appreciated that such sensors provide relatively higher resolution (higher sampling frequency) than the portable, non-lab-based systems (using commercial off the shelf (COTS) sensors). This allows for more metrics to be calculated from data gathered from lab-based systems than from systems using portable COTS components.

The subject-specific model 208 is tailored to a specific individual's baseline performance and physiological data from a selected set of measurements, which are a subset of the measurements that are captured in the lab. These selected measurements preferably come from wearable or portable sensors that can be deployed in real-world scenarios, as described above in FIG. 1. The system uses these selected physiological inputs to make real-time predictions about cognitive performance, such as creativity, fluid intelligence, and working memory. More specifically, the system predicts how an individual subject would have performed on an administered cognitive test. By utilizing both the general model 212 and the subject-specific model 218, the system may provide relatively reliable predictions even with a selected data set, making it viable for real-world applications. By way of example, a trained model herein, evaluating reading and rotational working memory tasks under cognitive loading, after 60 samples, indicated a prediction accuracy of 0.87, a precision of 0.9 and a recall of 0.8.

Aspects of the system herein can be embodied in programming. The technology herein may be considered as a product or article of manufacture in the form of a machine (processor) executable code and/or associated data that is carried on or embedded in a computer readable storage medium. The code can be pre-compiled and configured for use with the processor to execute the code or can be compiled during runtime.

More specifically, each dataset (lab-based dataset and selected in-house data set) preferably uses an 80/20 train-validation and test split. Five-fold cross validation of the train-validation set is used during model training and hyperparameter optimization. For feature selection, first, outliers are preferably clipped, constraining all predictor variables to lie within their 1st and 99th percentile bounds, thus mitigating the impact of outlier extremities. Next, a variance filter is preferably applied, whereby any feature exhibiting a variance below the 50-unit threshold is removed, as features with low variance are less likely to contribute meaningfully to the predictive model. Additionally, features that show a relatively minimal linear correlation with the target, defined as an absolute correlation coefficient below 0.03, are also removed. The final stage of feature selection preferably eliminates features that are highly correlated with one another, cutting off those with a correlation coefficient above 0.85. This reduces overfitting and improves the interpretability and generalization of the resultant predictive model. Selected features may be used to train a model using a Random Forest (RF) classifier. This model predicts an individual's cognitive performance from physiological data. Though the original model was trained as a generalized model (trained on population data), subsequent models were trained on an individual's data, resulting in a personalized model to predict an individual's cognitive performance.

It may therefore be appreciated that the present invention stands directed at a system to measure cognitive performance of a subject. The system provides a plurality of stimuli to the subject to provide for cognitive loading, followed by a measurement of a plurality of physiological data. The system includes a processor to then evaluate cognitive performance based upon a subject-specific model and a general model, wherein the system then predicts the cognitive performance of the individual to the plurality of stimuli.

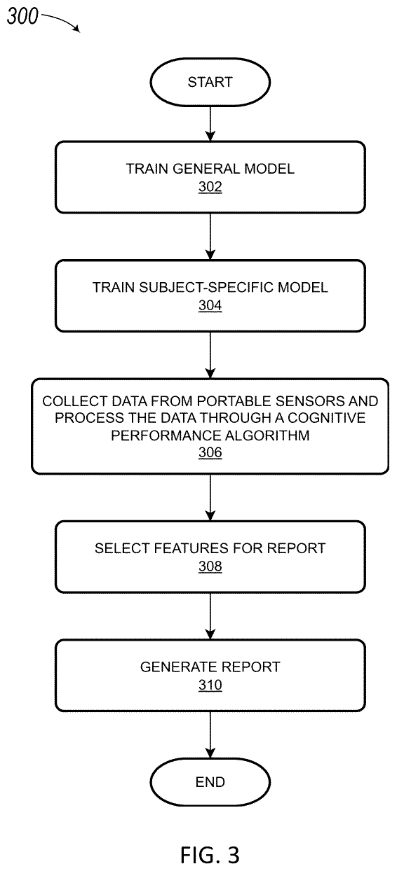

FIG. 3 is a flowchart diagram depicting operations for one illustrative example embodiment of the process 300 for assessment of cognitive performance on the system of FIG. 1, consistent with the present disclosure. It should be appreciated that embodiments of the present disclosure provide at least for the assessment of cognitive performance. However, FIG. 3 provides only an illustration of one implementation and does not imply any limitations with regard to the environments in which different embodiments may be implemented. Many modifications to the depicted environment may be made by those skilled in the art without departing from the scope of the disclosure as recited by the claims.

Process 300 includes training a general model (operation 302). In the illustrated example embodiment, the general model is trained using on data from a general population, as explained above. In an embodiment, the general model may be trained once and used repeatedly. In another embodiment, the general model may be trained once but updated from time to time when new data is available from the general population. In yet another embodiment, the general model may be trained for each operation of the system.

Process 300 includes training a subject-specific model (operation 304). The process 300 trains the subject-specific model using data from the specific subject, as explained above. In an embodiment, the subject-specific model may be trained once and used repeatedly. In another embodiment, the subject-specific model may be trained once but updated from time to time when new data is available from the specific subject. In yet another embodiment, the subject-specific may be trained for each operation of the system

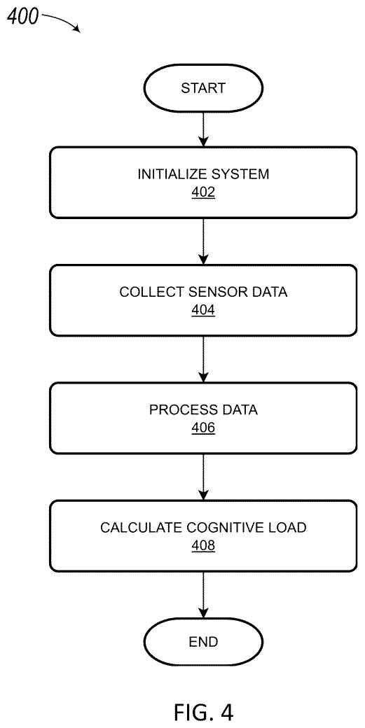

Process 300 includes collecting data from portable sensors and processing the data through a cognitive performance algorithm (operation 306). The operation for collecting data from portable sensors and processing the data through a cognitive performance algorithm is shown in FIG. 4.

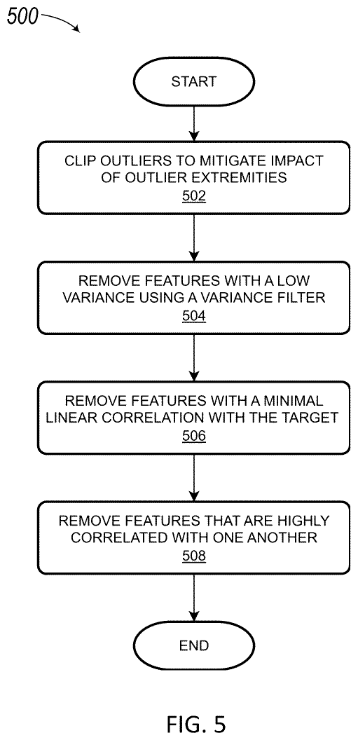

Process 300 includes selecting features for a report (operation 308). The process for selecting features for a report is shown in FIG. 5.

Process 300 includes generating the report (operation 310). The processes of operation 310 may include, but are not limited to, comparing the measured cognitive load to baseline levels, and providing insights into cognitive load variation. The results of operation 310 may include a real-time cognitive load report or visualization based on the features selected in operation 308.

Process 300 then returns the results to process 300 and ends for this cycle.

FIG. 4 is a flowchart diagram depicting operations for one illustrative example embodiment of the cognitive performance algorithm process 400 for the process 300 of FIG. 3, consistent with the present disclosure. It should be appreciated that embodiments of the present disclosure provide at least for the cognitive performance algorithm process 400. However, FIG. 4 provides only an illustration of one implementation and does not imply any limitations with regard to the environments in which different embodiments may be implemented. Many modifications to the depicted environment may be made by those skilled in the art without departing from the scope of the disclosure as recited by the claims.

Process 400 includes initializing the system (operation 402). In the illustrated example embodiment, the process 4000 activates and calibrates any necessary sensors. In an embodiment the necessary sensors may include, but are not limited to, a heart rate sensor, such as a chest strap, an eye tracker, and a speech recorder.

Process 400 includes collecting sensor data (operation 404). The process 400 collects sensor data that may include as an input the subject's real-time physiological data during activity/free state. The processes of operation 404 may include, but are not limited to, continuously collecting ECG data to monitor heart rate variability and changes, eye tracking data to measure gaze patterns, pupil dilation, microsaccades, etc., and combinations thereof, and speech data to analyze changes in voice patterns, tempo, pitch, etc., and combinations thereof, The results of operation 404 may include producing an output that includes, but is not limited to, real-time physiological data during non-specific cognitive activity.

Process 400 includes processing the sensor data (operation 406). The processes of operation 406 may include, but are not limited to, data cleaning, which may remove noise and artifacts from the raw physiological data; and feature extraction, which may extract relevant features from the heart rate data (e.g., heart rate variability), analyze the eye tracking data for metrics such as pupil dilation, gaze fixations, microsaccades, and combinations thereof, and evaluate the speech data for stress indicators like pitch variations, speech rate, and combinations thereof. The results of operation 406 may include, but are not limited to, cleaned and processed features from physiological data.

Process 400 includes calculating the cognitive load (operation 408). The processes of operation 408 may include, but are not limited to, applying the pre-trained subject-specific model to evaluate the processed features, and a cognitive load index calculation, which may integrate the extracted features using the subject-specific model to calculate a cognitive load index. The results of operation 408 may include the cognitive load index calculated based on the sensor measurements.

The process 400 then returns the results to process 300 and ends for this cycle.

FIG. 5 is a flowchart diagram depicting operations for one illustrative example embodiment of the feature selection process 500 for the process 300 of FIG. 3, consistent with the present disclosure. It should be appreciated that embodiments of the present disclosure provide at least for the feature selection process 500. However, FIG. 5 provides only an illustration of one implementation and does not imply any limitations with regard to the environments in which different embodiments may be implemented. Many modifications to the depicted environment may be made by those skilled in the art without departing from the scope of the disclosure as recited by the claims.

Process 500 includes clipping outliers to mitigate the impact of outlier extremities (operation 502). In the illustrated example embodiment, outliers are preferably clipped, constraining all predictor variables to lie within their 1st and 99th percentile bounds, thus mitigating the impact of outlier extremities.

Process 500 includes collecting sensor data (operation 504). Since features with low variance are less likely to contribute meaningfully to the predictive model, in operation 504 the process 500 removes features with a low variance using a variance filter. In an embodiment, any feature exhibiting a variance below a 50-unit threshold is removed.

Process 500 includes removing features with a minimal linear correlation with the target (operation 506). In an embodiment, the minimal linear correlation with the target may be defined as an absolute correlation coefficient below 0.03.

Process 500 includes removing features that are highly correlated with one another (operation 508). In an embodiment, the process 500 may cut off those features with a correlation coefficient above 0.85. This reduces overfitting and improves the interpretability and generalization of the resultant predictive model.

Process 500 then returns the results to process 300 and ends for this cycle.

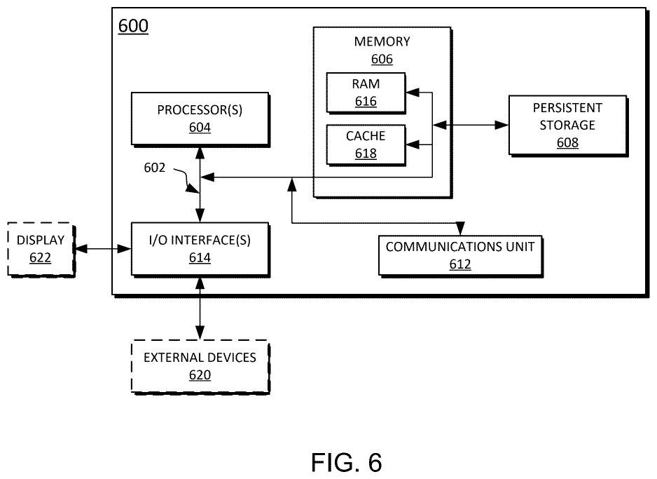

FIG. 6 is a block diagram depicting components of one example of the computing device 110 suitable for the assessment of cognitive performance, within the system of FIG. 1, consistent with the present disclosure. FIG. 6 displays the computing device or computer 600, one or more processor(s) 604 (including one or more controllers or computer processors), a communications fabric 602, a memory 606 including, a random-access memory (RAM) 616 and a cache 618, a persistent storage 608, a communications unit 612, I/O interfaces 614, a display 622, and external devices 620. It should be appreciated that FIG. 6 provides only an illustration of one embodiment and does not imply any limitations with regard to the environments in which different embodiments may be implemented. Many modifications to the depicted environment may be made.

As depicted, the computer 600 operates over the communications fabric 602, which provides communications between the computer processor(s) 604, memory 606, persistent storage 608, communications unit 612, and input/output (I/O) interface(s) 614. The communications fabric 602 may be implemented with an architecture suitable for passing data or control information between the processors 604 (e.g., microprocessors, communications processors, and network processors), the memory 606, the external devices 620, and any other hardware components within a system. For example, the communications fabric 602 may be implemented with one or more buses.

The memory 606 and persistent storage 608 are computer readable storage media. In the depicted embodiment, the memory 606 comprises a RAM 616 and a cache 618. In general, the memory 606 can include any suitable volatile or non-volatile computer readable storage media. Cache 618 is a fast memory that enhances the performance of processor(s) 604 by holding recently accessed data, and near recently accessed data, from RAM 616.

Program instructions for the assessment of cognitive performance may be stored in the persistent storage 608, or more generally, any non-transitory computer readable storage media, for execution by one or more of the respective computer processors 604 via one or more memories of the memory 606. The persistent storage 608 may be a magnetic hard disk drive, a solid-state disk drive, a semiconductor storage device, flash memory, read only memory (ROM), electronically erasable programmable read-only memory (EEPROM), or any other computer readable storage media that is capable of storing program instruction or digital information.

The media used by persistent storage 608 may also be removable. For example, a removable hard drive may be used for persistent storage 608. Other examples include optical and magnetic disks, thumb drives, and smart cards that are inserted into a drive for transfer onto another computer readable storage medium that is also part of persistent storage 608.

The communications unit 612, in these examples, provides for communications with other data processing systems or devices. In these examples, the communications unit 612 includes one or more network interface cards. The communications unit 612 may provide communications through the use of either or both physical and wireless communications links. In the context of some embodiments of the present disclosure, the source of the various input data may be physically remote to the computer 600 such that the input data may be received, and the output similarly transmitted via the communications unit 612.

The I/O interface(s) 614 allows for input and output of data with other devices that may be connected to computer 600. For example, the I/O interface(s) 614 may provide a connection to external device(s) 620 such as a keyboard, a keypad, a touch screen, a microphone, a digital camera, and/or some other suitable input device. External device(s) 620 can also include portable computer readable storage media such as, for example, thumb drives, portable optical or magnetic disks, and memory cards. Software and data used to practice embodiments of the present disclosure can be stored on such portable computer readable storage media and can be loaded onto persistent storage 608 via the I/O interface(s) 614.

I/O interface(s) 614 may also connect to a display 622. Display 622 provides a mechanism to display data to a user and may be, for example, a computer monitor. Display 622 can also function as a touchscreen, such as a display of a tablet computer.

According to one aspect of the disclosure there is thus provided a method for assessment of cognitive performance. The method includes attaching a plurality of portable physiological sensors to a body of a user; collecting data from the plurality of portable physiological sensors; processing the data using a cognitive performance algorithm and a pre-trained subject-specific model; and reporting a results of the cognitive performance algorithm.

According to another aspect of the disclosure, there is thus provided a system for assessment of cognitive performance. The system includes a pre-trained subject-specific model, a plurality of portable physiological sensors attached to a user; and a computing device. The system is configured to: collect data from the plurality of portable physiological sensors; process the data using a cognitive performance algorithm and the pre-trained subject-specific model; and report results of the cognitive performance algorithm.

According to yet another aspect of the disclosure, there is thus provided a non-transitory computer readable storage media including instructions that, when executed by processor circuitry, cause the processor circuitry to perform operations including: collect data from a plurality of portable physiological sensors, where the plurality of portable physiological sensors include at least one of: a chest-strap heartrate sensor; an eye tracking camera configured to measure eye microsaccades; and a head worn headset with a microphone; clean the data, where cleaning the data further comprises remove noise and artifacts from a raw physiological data; extract features from the data; process the data using a cognitive performance algorithm and a pre-trained subject-specific model; calculate a cognitive load, where calculate the cognitive load further comprises: apply the pre-trained subject-specific model to evaluate the extracted features; and calculate a cognitive load index, where the cognitive load index integrates the extracted features using the pre-trained subject-specific model; and report results of the cognitive performance algorithm.

Although the methods and systems have been described relative to a specific embodiment thereof, they are not so limited. Obviously, many modifications and variations may become apparent in light of the above teachings. Many additional changes in the details, materials, and arrangement of parts, herein described and illustrated, may be made by those skilled in the art. Also, it may be appreciated that the phraseology and terminology used herein is for the purpose of description and should not be regarded as limiting as such may be understood by one of skill in the art. Throughout the present disclosure, like reference characters may indicate like structure throughout the several views, and such structure need not be separately discussed. Furthermore, any particular feature(s) of a particular exemplary embodiment may be equally applied to any other exemplary embodiment(s) of this disclosure as suitable. In other words, features between the various exemplary embodiments described herein are interchangeable, and not exclusive.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

The term “coupled” as used herein refers to any connection, coupling, link, or the like by which signals carried by one system element are imparted to the “coupled” element. Such “coupled” devices, or signals and devices, are not necessarily directly connected to one another and may be separated by intermediate components or devices that may manipulate or modify such signals.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems. Throughout the entirety of the present disclosure, use of the articles “a” and/or “an” and/or “the” to modify a noun may be understood to be used for convenience and to include one, or more than one, of the modified noun, unless otherwise specifically stated. The terms “comprising”, “including” and “having” are intended to be inclusive and mean that there may be additional elements other than the listed elements.

The programs described herein are identified based upon the application for which they are implemented in a specific embodiment of the disclosure. However, it should be appreciated that any particular program nomenclature herein is used merely for convenience, and thus the disclosure should not be limited to use solely in any specific application identified and/or implied by such nomenclature.

The present disclosure may be a system, a method, and/or a computer program product. The system or computer program product may include one or more non-transitory computer readable storage media having computer readable program instructions thereon for causing a processor to carry out aspects of the present disclosure.

The one or more non-transitory computer readable storage media can be any tangible device that can retain and store instructions for use by an instruction execution device. The one or more non-transitory computer readable storage media may be, for example, but is not limited to, an electronic storage device, a magnetic storage device, an optical storage device, an electromagnetic storage device, a semiconductor storage device, or any suitable combination of the foregoing. A non-transitory computer readable storage media, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from one or more non-transitory computer readable storage media or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in one or more non-transitory computer readable storage media within the respective computing/processing device.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or a WAN, or the connection may be made to an external computer (for example, through the Internet using an Internet Service Provider). In some embodiments, electronic circuitry including, for example, programmable logic circuitry, Field-Programmable Gate Arrays (FPGA), or other Programmable Logic Devices (PLD) may execute the computer readable program instructions by utilizing state information of the computer readable program instructions to personalize the electronic circuitry, in order to perform aspects of the present disclosure.

It will be appreciated by those skilled in the art that any block diagrams herein represent conceptual views of illustrative circuitry embodying the principles of the disclosure. Similarly, it will be appreciated that any block diagrams, flow charts, flow diagrams, state transition diagrams, pseudocode, and the like represent various processes which may be substantially represented in computer readable medium and so executed by a computer or processor, whether or not such computer or processor is explicitly shown. Software modules, or simply modules which are implied to be software, may be represented herein as any combination of flowchart elements or other elements indicating performance of process steps and/or textual description. Such modules may be executed by hardware that is expressly or implicitly shown.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for assessment of cognitive performance, the method comprising:
attaching a plurality of portable physiological sensors to a body of a user;
collecting data for the user from the plurality of portable physiological sensors;
processing the data collected from the user using a cognitive performance algorithm and a pre-trained subject-specific model, wherein the pre-trained subject-specific model is trained with data from the user; and
reporting a result of the cognitive performance algorithm.

2. The method of claim 1, wherein processing the data using the cognitive performance algorithm further comprises:
cleaning the data collected from the user; and
extracting features from the data collected from the user.

3. The method of claim 2, further comprising:
calculating a cognitive load, wherein calculating the cognitive load further comprises:
applying the pre-trained subject-specific model to evaluate the extracted features; and
calculating a cognitive load index, wherein the cognitive load index integrates the extracted features using the pre-trained subject-specific model.

4. The method of claim 2, wherein cleaning the data collected from the user further comprises:
removing noise and artifacts from a raw physiological data.

5. The method of claim 4, further comprising:
calculating a cognitive load index based on the data collected from the user.

6. The method of claim 2, wherein extracting the features from the data collected from the user further comprises:
extracting one or more relevant features from the data, wherein the one or more relevant features include at least one of:
heart rate variability;
eye tracking data; and
speech data.

7. The method of claim 6, wherein the eye tracking data further comprises:
one or more eye metrics, the one or more eye metrics including at least one of pupil dilation, gaze fixations, microsaccades, or combinations thereof.

8. The method of claim 6, wherein the speech data further comprises:
one or more stress indicators, the one or more stress indicators including at least one of pitch variations, speech rate, or combinations thereof.

9. A system for assessment of cognitive performance, the system comprising:
a pre-trained subject-specific model, wherein the pre-trained subject-specific model is trained for a specific user;
a plurality of portable physiological sensors; and
a computing device;
the computing device configured to:
collect data from the plurality of portable physiological sensors attached to the specific user;
process the data using a cognitive performance algorithm and the pre-trained subject-specific model; and
report a result of the cognitive performance algorithm.

10. The system of claim 9, wherein the plurality of portable physiological sensors include at least one of:
a chest-strap heartrate sensor configured to measure a heart rate variability;
an eye tracking camera configured to measure eye microsaccades; and
a head worn headset with a microphone.

11. The system of claim 10, wherein the eye tracking camera is a near infrared vision camera.

12. The system of claim 9, wherein process the data using the cognitive performance algorithm further comprises:
clean the data; and
extract features from the data.

13. The system of claim 12, wherein cleaning the data further comprises:
removing noise and artifacts from a raw physiological data.

14. The system of claim 13, further comprising:
calculating a cognitive load index based on the data collected from the plurality of the portable physiological sensors.

15. The system of claim 12, wherein extracting the features from the data further comprises:
extracting one or more relevant features from the data, wherein the one or more relevant features include at least one of:
heart rate variability;
eye tracking data; and
speech data.

16. The system of claim 15, wherein the eye tracking data further comprises:
one or more eye metrics, the one or more eye metrics including at least one of pupil dilation, gaze fixations, microsaccades, and combinations thereof.

17. The system of claim 15, wherein the speech data further comprises:
one or more stress indicators, the one or more stress indicators including at least one of pitch variations and speech rate.

18. The system of claim 12, further comprising:
calculating a cognitive load, wherein calculating the cognitive load further comprises:
applying the pre-trained subject-specific model to evaluate the extracted features; and
calculating a cognitive load index, wherein the cognitive load index integrates the extracted features using the pre-trained subject-specific model.

19. A non-transitory computer readable storage media including instructions that, when executed by processor circuitry, cause the processor circuitry to perform operations comprising:
collect data from a plurality of portable physiological sensors attached to a user, wherein the plurality of portable physiological sensors include at least one of:
a chest-strap heartrate sensor;
an eye tracking camera configured to measure eye microsaccades;
a head worn headset with a microphone;
clean the data collected from the user, wherein cleaning the data further comprises remove noise and artifacts from a raw physiological data;
extract features from the data collected from the user;
process the data collected from the user using a cognitive performance algorithm and a pre-trained subject-specific model;
calculate a cognitive load for the user, wherein calculate the cognitive load for the user further comprises:
apply the pre-trained subject-specific model to the data collected from the user to evaluate the extracted features; and
calculate a cognitive load index for the user, wherein the cognitive load index integrates the extracted features using the pre-trained subject-specific model; and
report a result of the cognitive performance algorithm.

20. The non-transitory computer readable storage media of claim 19 including instructions that, when executed by the processor circuitry, cause the processor circuitry to perform operations further comprising:
extract one or more relevant features from the data, wherein the one or more relevant features include at least one of:
heart rate variability;
eye tracking data including one or more eye metrics, the one or more eye metrics including at least one of pupil dilation, gaze fixations, microsaccades, and combinations thereof; or
speech data, the speech data including one or more stress indicators, the one or more stress indicators including at least one of pitch variations, speech rate, and combinations thereof.
